(19) United States
(12) Patent Application Publication
(10) Pub. No.: US 2023/0404812 A1
(43) Pub. Date: Dec. 21, 2023

(54) EXTENDED WEAR DRESSING WITH SLOUGH CLEANING HOLES

(71) Applicant: KCI Manufacturing Unlimited Company, Westmeath (IE)

(72) Inventors: Shannon C. INGRAM, Bulverde, TX (US); Bradley JONIETZ, San Antonio, TX (US)

(21) Appl. No.: 18/035,902

(22) PCT Filed: Nov. 10, 2021
(86) PCT No.: PCT/IB2021/060396

§ 371 Date: May 8, 2023

Related U.S. Application Data
(60) Provisional application No. 63/112,240, filed on Nov. 11, 2020.

(51) Int. Cl. A61F 13/00; A61M 1/00

(52) U.S. Cl. CPC A61F 13/00068; A61F 13/00029; A61F 13/00034; A61M 1/915

(57) ABSTRACT

Dressings, systems, and methods for treating a tissue site are described. The dressing can include a first layer having a first side and a second side and a plurality of through-holes extending through the first layer from the first side to the second side. The dressing can also include a second layer configured to be positioned adjacent the second side of the first layer. The second layer can have a plurality of fluid restrictions and a plurality of perforations disposed in the second layer. The plurality of perforations can be configured to be aligned with the plurality of through-holes in the first layer.

### CROSS-REFERENCE TO RELATED APPLICATIONS

This application claims the benefit of priority to U.S. Provisional Application No. 63/112,240, filed on Nov. 11, 2020, which is incorporated herein by reference in its entirety.

### TECHNICAL FIELD

The invention set forth in the appended claims relates generally to tissue treatment systems and more particularly, but without limitation, to dressings for tissue treatment and methods of using the dressings for tissue treatment.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

For example, in some embodiments, a dressing for treating a tissue site is described. The dressing can include a first layer having a first side and a second side. The first layer can include a plurality of through-holes extending through the first layer from the first side to the second side. The dressing can include a second layer configured to be positioned adjacent the first side of the first layer. The second layer can include a plurality of fluid restriction disposed in the second layer. The second layer can also include a plurality of perforations disposed in the second layer and configured to be aligned with the plurality of through-holes.

A method of manufacturing a dressing for a tissue site is also described herein, wherein some example embodiments include providing a first layer and forming a plurality of through-holes in the first layer. A second layer can be provided, and a plurality of fluid restrictions and a plurality of perforation can be formed in the second layer. The second layer can be positioned adjacent the first layer.

Alternatively, other example embodiments may describe a system for treating a tissue site with reduced pressure. The system can include a dressing, a sealing member configured to be positioned over the dressing and sealed to tissue surrounding the tissue site, and a reduced-pressure source configured to be fluidly coupled to the dressing through the sealing member. The dressing can include a debridement tool having a first side and a second side, and a contact layer configured to be positioned adjacent the first side of the debridement tool. The debridement tool can include a plurality of openings extending through the debridement tool from the first side to the second side. The contact layer can include a plurality of fenestrations and a plurality of apertures. The plurality of apertures can be configured to be aligned with the plurality of openings.

A method of treating a tissue site is also described herein, wherein some example embodiments include positioning a dressing at the tissue site. The dressing can include a first layer and a second layer. The first layer can have a first side and a second side, and the second layer can be configured to be positioned adjacent the first side of the first layer. The first layer may have a plurality of through-holes extending through the first layer from the first side to the second side. The second layer may have a plurality of fluid restriction and a plurality of perforations. The plurality of perforations can be configured to be aligned with the plurality of through-holes. A negative-pressure source can be fluidly coupled to the dressing and negative pressure from the negative-pressure source can be applied to the dressing. A periphery of each of the plurality of perforations can stretch into the plurality of through-holes, creating a port through the first layer. Fluid may be drawn through the port.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment to a tissue site in accordance with this specification;

FIG. 2 is an assembly view of an example of a tissue interface of FIG. 1, illustrating additional details that may be associated with some embodiments;

FIG. 3 is a plan view of a first layer of the tissue interface of FIG. 2, illustrating additional details that may be associated with some embodiments;

FIG. 4 is a detail view of the first layer taken at Reference FIG. 4 in FIG. 3, illustrating additional details that may be associated with some embodiments;

FIG. 5 is a plan view of a second layer of the tissue interface of FIG. 2, illustrating additional details that may be associated with some embodiments;

FIG. 6 is a detail view of the second layer taken at reference FIG. 6 in FIG. 5, illustrating additional details that may be associated with some embodiments;

FIG. 7 is a detail view of the second layer taken at Reference FIG. 7 in FIG. 6, illustrating additional details that may be associated with some embodiments;

FIG. 8 is a cross-sectional view of the assembled tissue interface of FIG. 2 along line 8-8, illustrating additional details that may be associated with some embodiments;

FIG. 9 is a cross-sectional view of the assembled tissue interface of FIG. 2 along line 8-8 during negative-pressure therapy, illustrating additional details that may be associated with some embodiments;

FIG. 10 is a detail view of a portion of the tissue interface of FIG. 9, illustrating additional details of the operation of the tissue interface during negative-pressure therapy;

FIG. 11 is a perspective view of the second layer of FIG. 2, illustrating additional details that may be associated with some embodiments;

FIG. 12 is a cross-section view of the second layer of FIG. 11 along line 12-12, illustrating additional details that may be associated with some embodiments;

FIG. 13 is an assembly view of another example of the tissue interface of FIG. 1, illustrating additional details that may be associated with some embodiments; and

FIG. 14 is a cross-sectional view of the assembled tissue interface of FIG. 13 along line 14-14, illustrating additional details that may be associated with some embodiments.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

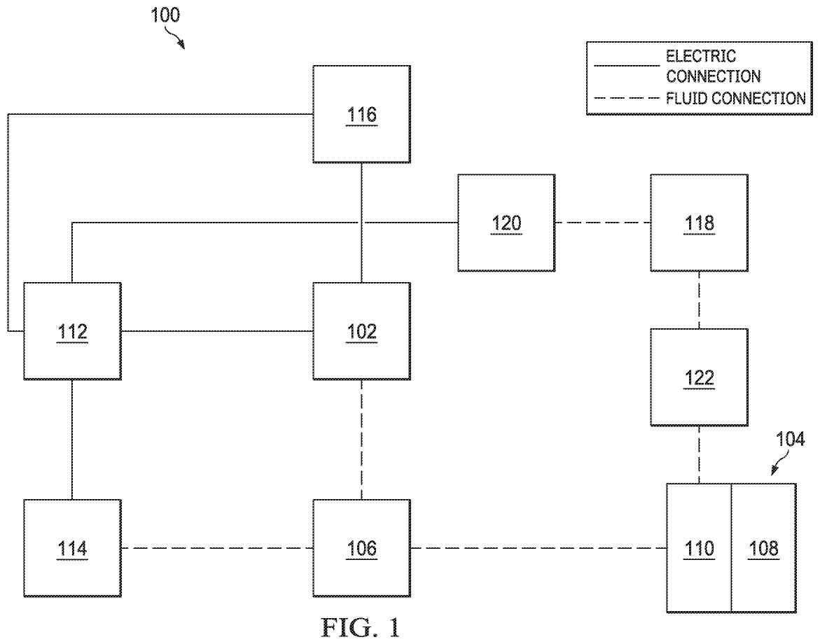

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including but not limited to, a surface wound, bone tissue, adipose tissue, muscle tissue, neural tissue, dermal tissue, vascular tissue, connective tissue, cartilage, tendons, or ligaments. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted. A surface wound, as used herein, is a wound on the surface of a body that is exposed to the outer surface of the body, such as injury or damage to the epidermis, dermis, and/or subcutaneous layers. Surface wounds may include ulcers or closed incisions, for example. A surface wound, as used herein, does not include wounds within an intra-abdominal cavity. A wound may include chronic, acute, traumatic, subacute, and dehisced wounds, partial-thickness burns, ulcers (such as diabetic, pressure, or venous insufficiency ulcers), flaps, and grafts, for example.

The therapy system 100 may include a source or supply of negative pressure, such as a negative-pressure source 102, and one or more distribution components. A distribution component is preferably detachable and may be disposable, reusable, or recyclable. A dressing, such as a dressing 104, and a fluid container, such as a container 106, are examples of distribution components that may be associated with some examples of the therapy system 100. As illustrated in the example of FIG. 1, the dressing 104 may comprise or consist essentially of a tissue interface 108, a cover 110, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104. For example, such a dressing interface may be a SENSAT.RA.C.™ Pad available from Kinetic Concepts, Inc. of San Antonio, Texas.

The therapy system 100 may also include a regulator or controller, such as a controller 112. Additionally, the therapy system 100 may include sensors to measure operating parameters and provide feedback signals to the controller 112 indicative of the operating parameters. As illustrated in FIG. 1, for example, the therapy system 100 may include a first sensor 114 and a second sensor 116 coupled to the controller 112.

The therapy system 100 may also include a source of instillation solution. For example, a solution source 118 may be fluidly coupled to the dressing 104, as illustrated in the example embodiment of FIG. 1. The solution source 118 may be fluidly coupled to a positive-pressure source, such as a positive-pressure source 120, a negative-pressure source, such as the negative-pressure source 102, or both in some embodiments. A regulator, such as an instillation regulator 122, may also be fluidly coupled to the solution source 118 and the dressing 104 to ensure proper dosage of instillation solution (e.g. saline) to a tissue site. For example, the instillation regulator 122 may comprise a piston that can be pneumatically actuated by the negative-pressure source 102 to draw instillation solution from the solution source during a negative-pressure interval and to instill the solution to a dressing during a venting interval. Additionally or alternatively, the controller 112 may be coupled to the negative-pressure source 102, the positive-pressure source 120, or both, to control dosage of instillation solution to a tissue site. In some embodiments, the instillation regulator 122 may also be fluidly coupled to the negative-pressure source 102 through the dressing 104.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the controller 112, the solution source 118, and other components into a therapy unit.

In general, components of the therapy system 100 may be coupled directly or indirectly. For example, the negative-pressure source 102 may be directly coupled to the container 106 and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to the controller 112 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 102, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 102 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy. In some embodiments, the container 106 may comprise a canister having a collection chamber, a first inlet fluidly coupled to the collection chamber and a first outlet fluidly coupled to the collection chamber and adapted to receive negative pressure from a source of negative pressure.

A controller, such as the controller 112, may be a microprocessor or computer programmed to operate one or more components of the therapy system 100, such as the negative-pressure source 102. In some embodiments, for example, the controller 112 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 108, for example. The controller 112 is also preferably configured to receive one or more input signals, such as a feedback signal, and programmed to modify one or more operating parameters based on the input signals.

Sensors, such as the first sensor 114 and the second sensor 116, are generally known in the art as any apparatus operable to detect or measure a physical phenomenon or property, and generally provide a signal indicative of the phenomenon or property that is detected or measured. For example, the first sensor 114 and the second sensor 116 may be configured to measure one or more operating parameters of the therapy system 100. In some embodiments, the first sensor 114 may be a transducer configured to measure pressure in a pneumatic pathway and convert the measurement to a signal indicative of the pressure measured. In some embodiments, for example, the first sensor 114 may be a piezo-resistive strain gauge. The second sensor 116 may optionally measure operating parameters of the negative-pressure source 102, such as a voltage or current, in some embodiments. Preferably, the signals from the first sensor 114 and the second sensor 116 are suitable as an input signal to the controller 112, but some signal conditioning may be appropriate in some embodiments. For example, the signal may need to be filtered or amplified before it can be processed by the controller 112. Typically, the signal is an electrical signal, but may be represented in other forms, such as an optical signal.

The tissue interface 108 can be generally adapted to partially or fully contact a tissue site. The tissue interface 108 may take many forms, and may have many sizes, shapes, or thicknesses, depending on a variety of factors, such as the type of treatment being implemented or the nature and size of a tissue site. The tissue interface 108 may further promote granulation at a tissue site when pressure within the sealed therapeutic environment is reduced. For example, the size and shape of the tissue interface 108 may be adapted to the contours of deep and irregular shaped tissue sites. Any or all of the surfaces of the tissue interface 108 may have an uneven, coarse, or jagged profile that can induce microstrains and stresses at a tissue site if negative pressure is applied through the tissue interface 108.

In some embodiments, the tissue interface 108 may comprise or consist essentially of a manifold. A manifold in this context may comprise or consist essentially of a means for collecting or distributing fluid across the tissue interface 108 under pressure. For example, a manifold may be adapted to receive negative pressure from a source and distribute negative pressure through multiple apertures across the tissue interface 108, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 110 may provide a bacterial barrier and protection from physical trauma. The cover 110 may also be constructed from a material that can reduce evaporative losses and provide a fluid seal between two components or two environments, such as between a therapeutic environment and a local external environment. The cover 110 may comprise or consist of, for example, an elastomeric film or membrane that can provide a seal adequate to maintain a negative pressure at a tissue site for a given negative-pressure source. The cover 110 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 110 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S. A., Colombes, France; and Inspire 2301 and Inpsire 2327 polyurethane films, commercially available from Expopack Advanced Coatings, Wrexham, United Kingdom. In some embodiments, the cover 110 may comprise INSPIRE 2301 having an MVTR (upright cup technique) of 2600 g/m²/24 hours and a thickness of about 30 microns.

An attachment device may be used to attach the cover 110 to an attachment surface, such as undamaged epidermis, a gasket, or another cover. The attachment device may take many forms. For example, an attachment device may be a medically-acceptable, pressure-sensitive adhesive configured to bond the cover 110 to epidermis around a tissue site. In some embodiments, for example, some or all of the cover 110 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 118 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 108 may be placed within, over, on, or otherwise proximate to a tissue site. If the tissue site is a wound, for example, the tissue interface 108 may partially or completely fill the wound, or it may be placed over the wound. The cover 110 may be placed over the tissue interface 108 and sealed to an attachment surface near a tissue site. For example, the cover 110 may be sealed to undamaged epidermis peripheral to a tissue site. Thus, the dressing 104 can provide a sealed therapeutic environment proximate to a tissue site, substantially isolated from the external environment, and the negative-pressure source 102 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 108 in the sealed therapeutic environment can induce macro-strain and micro-strain in the tissue site. Negative pressure can also remove exudate and other fluid from a tissue site, which can be collected in container 106.

In some embodiments, the controller 112 may receive and process data from one or more sensors, such as the first sensor 114. The controller 112 may also control the operation of one or more components of the therapy system 100 to manage the pressure delivered to the tissue interface 108. In some embodiments, controller 112 may include an input for receiving a desired target pressure and may be programmed for processing data relating to the setting and inputting of the target pressure to be applied to the tissue interface 108. In some example embodiments, the target pressure may be a fixed pressure value set by an operator as the target negative pressure desired for therapy at a tissue site and then provided as input to the controller 112. The target pressure may vary from tissue site to tissue site based on the type of tissue forming a tissue site, the type of injury or wound (if any), the medical condition of the patient, and the preference of the attending physician. After selecting a desired target pressure, the controller 112 can operate the negative-pressure source 102 in one or more control modes based on the target pressure and may receive feedback from one or more sensors to maintain the target pressure at the tissue interface 108.

During treatment of a tissue site, some tissue sites may not heal according to the normal medical protocol and may develop areas of necrotic tissue. Necrotic tissue may be dead tissue resulting from infection, toxins, or trauma that caused the tissue to die faster than the tissue can be removed by the normal body processes that regulate the removal of dead tissue. Sometimes, necrotic tissue may be in the form of slough, which may include a viscous liquid mass of tissue. Generally, slough is produced by bacterial and fungal infections that stimulate an inflammatory response in the tissue. Slough may be a creamy yellow color and may also be referred to as pus. Necrotic tissue may also include eschar. Eschar may be a portion of necrotic tissue that has become dehydrated and hardened. Eschar may be the result of a burn injury, gangrene, ulcers, fungal infections, spider bites, or anthrax. Eschar may be difficult to remove without the use of surgical cutting instruments.

In addition to necrotic tissue, slough, and eschar, the tissue site may include biofilms, lacerated tissue, devitalized tissue, contaminated tissue, damaged tissue, infected tissue, exudate, highly viscous exudate, fibrinous slough and/or other material that can generally be referred to as debris. The debris may inhibit the efficacy of tissue treatment and slow the healing of the tissue site. If the debris is in the tissue site, the tissue site may be treated with different processes to disrupt the debris. Examples of disruption can include softening of the debris, separation of the debris from desired tissue, such as the subcutaneous tissue, preparation of the debris for removal from the tissue site, and removal of the debris from the tissue site.

The debris can require debridement performed in an operating room. In some cases, tissue sites requiring debridement may not be life-threatening, and debridement may be considered low-priority. Low-priority cases can experience delays prior to treatment as other, more life-threatening, cases may be given priority for an operating room. As a result, low priority cases may need temporization. Temporization can include stasis of a tissue site that limits deterioration of the tissue site prior to other treatments, such as debridement, negative-pressure therapy or instillation.

When debriding, clinicians may find it difficult to define separation between healthy, vital tissue and necrotic tissue. As a result, normal debridement techniques may remove too much healthy tissue or not enough necrotic tissue. If non-viable tissue demarcation does not extend deeper than the deep dermal layer, or if the tissue site is covered by the debris, such as slough or fibrin, gentle methods to remove the debris should be considered to avoid excess damage to the tissue site.

In some debridement processes, a mechanical process is used to remove the debris. Mechanical processes may include using scalpels or other cutting tools having a sharp edge to cut away the debris from the tissue site. Other mechanical processes may use devices that can provide a stream of particles to impact the debris to remove the debris in an abrasion process, or jets of high pressure fluid to impact the debris to remove the debris using water-jet cutting or lavage. Typically, mechanical processes of debriding a tissue site may be painful and may require the application of local anesthetics. Mechanical processes also risk over removal of healthy tissue that can cause further damage to the tissue site and delay the healing process. In some debridement processes, a dressing that may be covering a tissue site is generally removed to permit access to the tissue site for debridement, preventing use of a dressing for an extended wear time. Thus, patients needing debridement may not receive the benefits of associated with the protection and therapy provided by wearing the dressing for a medically beneficial period.

Debridement may also be performed with an autolytic process. For example, an autolytic process may involve using enzymes and moisture produced by a tissue site to soften and liquefy the necrotic tissue and debris. Typically, a dressing may be placed over a tissue site having debris so that fluid produced by the tissue site may remain in place, hydrating the debris. Autolytic processes can be pain-free, but autolytic processes are a slow and can take many days. Because autolytic processes are slow, autolytic processes may also involve many dressing changes. Some autolytic processes may be paired with negative-pressure therapy so that, as debris hydrates, negative pressure supplied to a tissue site may draw off the debris. In some cases, a manifold positioned at a tissue site to distribute negative-pressure across the tissue site may become blocked or clogged with debris broken down by an autolytic process. If a manifold becomes clogged, negative-pressure may not be able to remove debris, which can slow or stop the autolytic process. Additionally, if the manifold is left at a tissue site for an extended period of time, for example, to aid in healing of the tissue site as the debris is removed, ingrowth of granulation tissue may become a concern.

Debridement may also be performed by adding enzymes or other agents to the tissue site that digest tissue. Often, strict control of the placement of the enzymes and the length of time the enzymes are in contact with a tissue site must be maintained. If enzymes are left on a tissue site for longer than needed, the enzymes may remove too much healthy tissue, contaminate the tissue site, or be carried to other areas of a patient. Once carried to other areas of a patient, the enzymes may break down undamaged tissue and cause other complications.

These limitations and others may be addressed by the therapy system 100, which can provide negative-pressure therapy, instillation therapy, and disruption of debris. In some embodiments, the therapy system 100 can provide mechanical movement at a surface of the tissue site in combination with cyclic delivery and dwell of topical solutions to help solubilize debris. For example, a negative-pressure source may be fluidly coupled to a tissue site to provide negative pressure to the tissue site for negative-pressure therapy. In some embodiments, a fluid source may be fluidly coupled to a tissue site to provide therapeutic fluid to the tissue site for instillation therapy. In some embodiments, the therapy system 100 may include a contact layer positioned adjacent to a tissue site that may be used with negative-pressure therapy, instillation therapy, or both to disrupt areas of a tissue site having debris. Following the disruption of the debris, negative-pressure therapy, instillation therapy, and other processes may be used to remove the debris from a tissue site. In some embodiments, the therapy system 100 may be used in conjunction with other tissue removal and debridement techniques. For example, the therapy system 100 may be used prior to enzymatic debridement to soften the debris. In another example, mechanical debridement may be used to remove a portion of the debris at the tissue site, and the therapy system 100 may then be used to remove the remaining debris while reducing the risk of trauma to the tissue site. The therapy system 100 may also provide a dressing that can provide debridement of tissue while being worn for an extended period of time and preventing granulation tissue in-growth. Dressings that can be worn for extended periods can provide cost-savings, time-efficiencies, and less trauma to a patient during dressing changes.

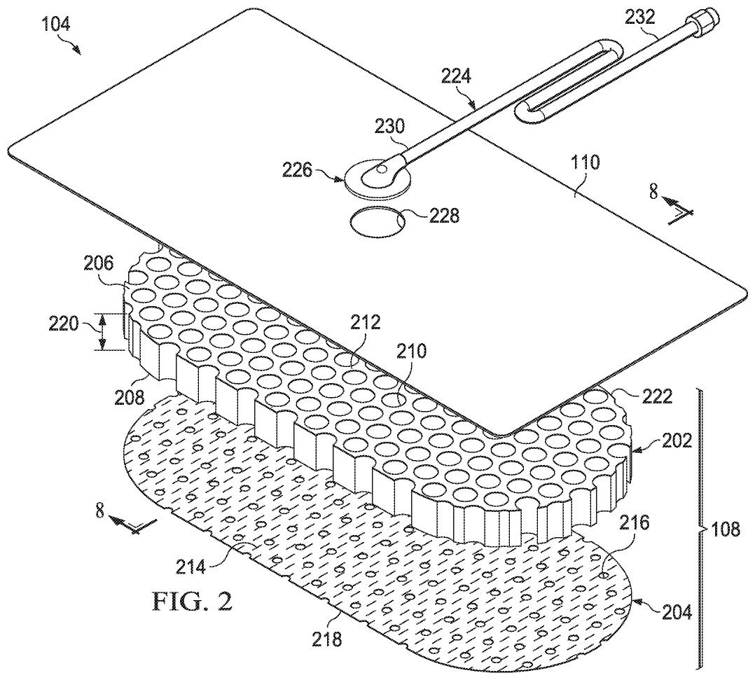

FIG. 2 is an assembly view of an example of the dressing 104 of FIG. 1, illustrating additional details that may be associated with some embodiments. The dressing 104 may comprise the tissue interface 108 and the cover 110. In some embodiments, the tissue interface 108 may include a manifold or a debridement tool, such as a first layer 202, and a contact layer, such as a second layer 204. The first layer may comprise a first side 206 and a second side 208 opposite the first side 206. The first layer 202 may have a substantially uniform thickness 220 extending from the first side 206 to the second side 208. In some embodiments, the thickness 220 of the first layer 2020 may be between about 2 millimeters and about 12 millimeters. In other embodiments, the thickness 220 of the first layer 202 may be selected for a particular application of the tissue interface 108. The second layer 204 may also be flexible so that the second layer 204 can be contoured to a surface of the tissue site.

In some embodiments, the first layer 202 may comprise a foam. For example, the first layer 202 may be a reticulated foam having pore sizes and free volume that may vary according to needs of a prescribed therapy. For example, a reticulated foam having a free volume of at least 90% may be suitable for many therapy applications, and a foam having an average pore size in a range of 400-600 microns may be particularly suitable for some types of therapy. The tensile strength of the first layer 202 may also vary according to needs of a prescribed therapy. For example, the tensile strength of a foam may be increased for instillation of topical treatment solutions. The 25% compression load deflection of the first layer 202 may be at least 2.2 pounds per square inch. In some embodiments, the tensile strength of the first layer 202 may be at least 18 pounds per square inch. The first layer 202 may have a tear strength of at least 4 pounds per inch. In some embodiments, the first layer 202 may be a foam comprised of polyols such as polyester or polyether, isocyanate such as toluene diisocyanate, and polymerization modifiers such as amines and tin compounds. In some examples, the first layer 202 may be a reticulated polyurethane foam such as used in GRANUFOAM™ Dressing or V.A.C. VERAFLO™ Dressing, both available from KCI of San Antonio, Texas.

In some embodiments, the first layer 202 may be formed from a foam that is mechanically or chemically compressed, often as part of a thermoforming process, to increase the density of the foam at ambient pressure. A foam that is mechanically or chemically compressed may be referred to as a compressed foam or a felted foam. In some embodiments, the first layer 202 may be formed by a felting process. Felting comprises a thermoforming process that permanently compresses a foam to increase the density of the foam while maintaining interconnected pathways. For example, felting may be performed by applying heat and pressure to a porous material or foam material. Some methods may include compressing a foam blank between one or more heated platens or dies (not shown) for a specified period of time and at a specified temperature. The direction of compression may be along the thickness of the foam blank.

The period of time of compression may range from 10 minutes up to 24 hours, though the time period may be more or less depending on the specific type of porous material used. Further, in some examples, the temperature may range between 120° C. to 260° C. Generally, the lower the temperature of the platen, the longer a porous material must be held in compression. After the specified time period has elapsed, the pressure and heat will form a felted structure or surface on or through the porous material or a portion of the porous material.

The felting process may alter certain properties of the original material, including pore shape and/or size, elasticity, density, and density distribution. For example, struts that define pores in the foam may be deformed during the felting process, resulting in flattened pore shapes. The deformed struts can also decrease the elasticity of the foam. The density of the foam is generally increased by felting. In some embodiments, contact with hot-press platens in the felting process can also result in a density gradient in which the density is greater at the surface and the pore size is smaller or eliminated at the surface. For example, at least a portion of the first layer 202 may be felted to the point of having no pores. In some embodiments, the felted structure may be comparatively smoother than any unfinished or non-felted surface or portion of the porous material. Further, the pores in the felted structure may be smaller than the pores throughout any unfinished or non-felted surface or portion of the porous material. In some examples, the felted structure may be applied to all surfaces or portions of the porous material. Further, in some examples, the felted structure may extend into or through an entire thickness of the porous material such that the all of the porous material is felted.

A felted foam may be characterized by a firmness factor, which is indicative of the compression of the foam. The firmness factor of a felted foam can be specified as the ratio of original thickness to final thickness. A compressed or felted foam may have a firmness factor greater than 1. The degree of compression may affect the physical properties of the felted foam. For example, felted foam has an increased effective density compared to a foam of the same material that is not felted. The felting process can also affect fluid-to-foam interactions. For example, as the density increases, compressibility or collapse may decrease. Therefore, foams which have different compressibility or collapse may have different firmness factors. In some example embodiments, a firmness factor can range from about 2 to about 10, preferably about 3 to about 5. For example, the firmness factor of the first layer 202 felted foam may be about 5 in some embodiments. There is a general linear relationship between firmness level, density, pore size (or pores per inch) and compressibility. For example, foam that is felted to a firmness factor of 3 will show a three-fold density increase and remain compressed to about a third of its original thickness.

In some embodiments, one or more suitable foam blanks (e.g. of pre-felted foam) may be used for forming the first layer 202. The foam blank(s) may have about 40 to about 150 pores per inch on average, a density of about 5.1 to about 6.3 lb/ft³, an average pore size in a range of about 133 to about 600 microns, a 25% compression load deflection of at least 2.2 pounds per square inch, and/or a 65% compression load deflection of at least 2.2 pounds per square inch. In some embodiments, the foam blank(s) may have a thickness greater than 10 millimeters. For example, the foam blank(s) may have a thickness in a range of about 10 and about 35 millimeters, about 10 and about 25 millimeters, about 10 and about 20 millimeters, or about 15 and about 20 millimeters. In some embodiments, the foam blank(s) may be felted to provide denser foam for the first layer 202. For example, one or more foam blanks may be felted to a firmness factor of 2-10 to form the first layer 202. In some embodiments, the foam blank may be felted to a firmness factor of 3-7. Some embodiments may felt the foam blank to a firmness factor of 5.

In some embodiments, the first layer 202 may be an open-cell foam having a free volume in a range of about 9% to about 45%, a density in a range of about 2.6 to about 16 lb/ft³, about 80 to about 500 pores per inch on average as measured in the direction of compression, an average pore size in a range of about 40 to about 300 microns as measured in the direction of compression, and/or a 25% compression load deflection of about 0.7 to about 3.5 pounds per square inch and a 65% compression load deflection of about 0.86 to about 4.3 pounds per square inch, which may be particularly advantageous under negative pressure. In some embodiments, the first layer 202 may be an open-cell foam having a free volume in a range of about 18% to about 45%, a density in a range of about 2.6 to about 8 lb/ft³, about 40 to about 250 pores per inch on average (e.g., as measured in the direction of compression), an average pore size in a range of about 80 to about 600 microns (e.g., as measured in the direction of compression), and/or a 25% compression load deflection of about 2.2 pounds per square inch and a 65% compression load deflection of about 2.2 pounds per square inch, which may be particularly advantageous under negative pressure.

As further shown in FIG. 2, the first layer 202 may include a plurality of through-holes 210 extending through the first layer 202 from the first side 206 to the second side 208. The plurality of through-holes 210 can be distributed uniformly or randomly across the first layer 202. The plurality of through-holes 210 extending through the first layer 202 may form walls 212 extending through the first layer 202. The through-holes 210 may have a depth that is about equal to the thickness 220 of the first layer 202. For example, the through-holes 210 may have a depth between about 2 millimeters and about 12 millimeters. In some embodiments the through-holes 210 may have a depth of about 8 millimeters.

In some embodiments, at least one of the plurality of through-holes 210 may be positioned at an edge of the first layer 202. For example, at least one of the plurality of through-holes 210 may be positioned at the periphery 222 of the first layer 202 and an interior portion of the through-hole 210 may be exposed. In some embodiments, the plurality of through-holes 210 positioned at or near the periphery 222 of the first layer 202 may be substantially equidistantly spaced around the periphery 222. Additionally or alternatively, the spacing of the plurality of through-holes 210 proximate to the periphery 222 of the first layer 202 may be irregular.

In some embodiments, the second layer 204 may be configured to be positioned adjacent the second side 208 of the first layer 202. For example, the second layer 204 and the first layer 202 may be stacked so that the second layer 204 is in contact with the first layer 202. In some embodiments, the second layer 204 may be coupled to the second side 208 of the first layer 202.

The second layer 204 may include a means for controlling or managing fluid flow. In some embodiments, the second layer 204 may be a fluid control layer comprising a liquid-impermeable, elastomeric material. For example, the second layer 204 may be a polymer film, such as a polyurethane film. In some embodiments, the second layer 204 may be the same material as the cover 110. The second layer 204 may also have a smooth or matte surface texture in some embodiments. A glossy or shiny finish finer or equal to a grade B3 according to the SPI (Society of the Plastics Industry) standards may be particularly advantageous for some applications. In some embodiments, variations in surface height may be limited to acceptable tolerances. For example, the surface of the second layer 204 may have a substantially flat surface, with height variations limited to 0.2 millimeters over a centimeter.

In some embodiments, the second layer 204 may be hydrophobic. The hydrophobicity of the second layer 204 may vary, but may have a contact angle with water of at least ninety degrees in some embodiments. In some embodiments, the second layer 204 may have a contact angle with water of no more than 150 degrees. For example, in some embodiments, the contact angle of the second layer 204 may be in a range of at least 90 degrees to about 120 degrees, or in a range of at least 120 degrees to 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things. Non-limiting examples of such integrated systems may include the FTÅ125, FTÅ200, FTÅ2000, and FTÅ4000 systems, all commercially available from First Ten Angstroms, Inc., of Portsmouth, VA, and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles herein represent averages of 5 to 9 measured values, discarding both the highest and lowest measured values. The hydrophobicity of the second layer 204 may be further enhanced with a hydrophobic coating of other materials, such as silicones and fluorocarbons, either as coated from a liquid, or plasma coated.

The second layer 204 may also be suitable for welding to other layers, including the first layer 202. For example, the second layer 204 may be adapted for welding to polyurethane foams using heat, radio frequency (RF) welding, or other methods to generate heat such as ultrasonic welding. RF welding may be particularly suitable for more polar materials, such as polyurethane, polyamides, polyesters and acrylates. Sacrificial polar interfaces may be used to facilitate RF welding of less polar film materials, such as polyethylene. In some embodiments, the second layer 204 may be flame laminated to the first layer 202.

The area density of the second layer 204 may vary according to a prescribed therapy or application. In some embodiments, an area density of less than 40 grams per square meter may be suitable, and an area density of about 20 to 30 grams per square meter may be particularly advantageous for some applications.

In some embodiments, for example, the second layer 204 may be a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between 20 microns and 100 microns may be suitable for many applications. Films may be clear, colored, or printed. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations.

The second layer 204 may have one or more sets of fluid passages, which can be distributed uniformly or randomly across the second layer 204. In some embodiments, the passages may be bi-directional and pressure-responsive. For example, each of the passages generally may be an elastic passage that is normally unstrained to substantially reduce liquid flow, and can expand or open in response to a pressure gradient and/or in response to the contraction of the first layer 202. In some embodiments, a plurality of fluid restrictions 214 may be disposed in the second layer 204. The plurality of fluid restrictions 214 may be formed by removing material from the second layer 204. For example, the plurality of fluid restrictions 214 may be formed by cutting through the second layer 204. In the absence of a pressure gradient across the plurality of fluid restrictions 214, the plurality of fluid restrictions 214 may be sufficiently small to form a seal, which can substantially reduce or prevent liquid flow. Additionally, or alternatively, one or more of the passages may be or may function as an elastomeric valve that is normally closed when unstrained to substantially prevent liquid flow, and can open in response to a pressure gradient and/or in response to the contraction of the first layer 202. In some embodiments, the plurality of fluid restrictions 214 may be fenestrations in the second layer 204. In some embodiments, at least a portion of the plurality of fluid restriction 214 may be registered with at least a portion of the plurality of through-holes 210 in the first layer 202.

In some embodiments, a plurality of perforations 216 may also be disposed in the second layer 204. The plurality of perforations 216 may be formed by cutting or by application of local RF or ultrasonic energy, for example, or by other suitable techniques for forming an opening. The plurality of perforations 216 in the second layer 204 may be configured to be aligned with the plurality of through-holes 210 in the first layer 202. In other embodiments, the plurality of perforations 216 may have a uniform distribution pattern, or may be randomly distributed on the second layer 204. The plurality of perforations 216 may also have many shapes, including circular, square, elliptical, polygonal, and amorphous shapes.

At least one of the plurality of perforations 216 may be positioned at an edge of the second layer 204. For example, at least one of the plurality of perforations 216 may be positioned at a periphery 218 of the second layer 204 and an interior portion of the perforation 216 may be exposed. The plurality of perforations 216 positioned at or near the periphery 218 of the second layer 204 may be substantially equidistantly spaced around the periphery 218. Additionally or alternatively, the spacing of the plurality of perforations 216 proximate to the periphery 218 of the second layer 204 may be irregular.

In some embodiments, the dressing 104 may include a fluid conductor 224 and a dressing interface 226. In some embodiments, the fluid conductor 224 may be a flexible tube. In some embodiments, the fluid conductor may comprise a first end 230 and a second end 232. The first end 230 of the fluid conductor 224 may be configured to be fluidly coupled to the dressing interface 226 and the second end 232 of the fluid conductor 224 may be configured to be fluidly coupled to the negative-pressure source 102 (not shown).

In some embodiments, the dressing interface 226 may be an elbow connector, as shown in the example of FIG. 2, which can be placed over an aperture 228 in the cover 110 to provide a fluid path between the fluid conductor 224 and the tissue interface 108. In other embodiments, the first end 230 of the fluid conductor 224 may be inserted directly through the cover 110 into the tissue interface 108. The cover 110 may be configured to create a sealed space containing the first layer 202 and the second layer 204 at the tissue site 806. In some embodiments, the cover 110 may include the aperture 228. In other embodiments, the aperture 228 may be cut into the cover 110 before or after positioning the cover 110 over the tissue interface 108. In some embodiments, the aperture 228 may be centrally disposed in the cover 110. In other embodiments, the position of the aperture 228 may be off-center or adjacent to an end or edge of the cover 110.

In some embodiments, the tissue interface 108 may be provided as a portion of an assembly forming the dressing 104. In other embodiments, the tissue interface 108 may be provided separately from the cover 110, the fluid conductor 224, and the dressing interface 226 for assembly of the dressing 104 at the point of use.

If not already configured, the dressing interface 226 may be disposed over the aperture 228 and attached to the cover 110. The first end 230 of the fluid conductor 224 may be fluidly coupled to the dressing interface 226 and the second end 232 of the fluid conductor 224 may be fluidly coupled to the negative-pressure source 102.

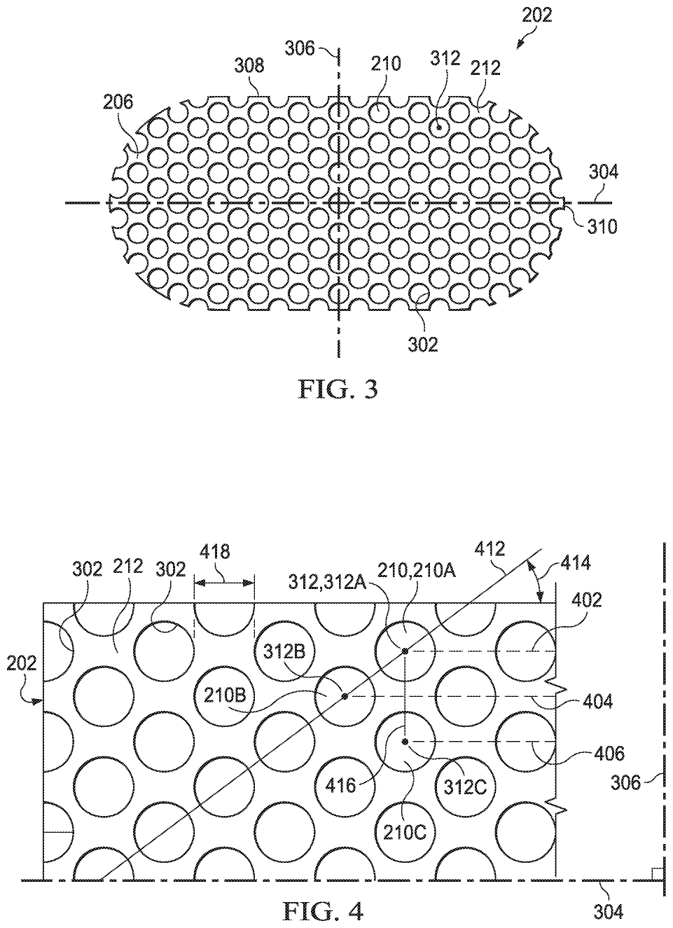

FIG. 3 is a plan view of the first layer 202 of FIG. 2, illustrating additional details that may be associated with some embodiments. The first layer 202 may include the plurality of through-holes 210 extending through the first layer 202 to form the walls 212. In some embodiments, the through-holes 210 may have a circular shape. In other embodiments, the through-holes 210 may have other shapes and orientations, for example, elliptical, hexagonal, square, triangular, polygonal, or amorphous or irregular.

In some embodiments, an interior surface of the walls 212 may be parallel to the thickness 220 of the first layer 202. In other embodiments, an interior surface of the walls 212 may be generally perpendicular to the first side 206 and the second side 208 of the first layer 202. The interior surface or surfaces of the walls 212 may form a perimeter 302 of each through-hole 210 and may connect the first side 206 to the second side 208. In other embodiments, the through-holes 210 may be formed so that the interior surfaces of the walls 212 of the through-holes 210 form a non-perpendicular angle with the first side 206. In still other embodiments, the interior surfaces of the walls 212 of the through-holes 210 may taper toward a center 312 of the through-holes 210 to form conical, pyramidal, or other irregular through-hole shapes. If the interior surfaces of the walls 212 of the through-holes 210 taper, the through-holes 210 may have a height less than the thickness 220 of the first layer 202.

In some embodiments, the first layer 202 may have a first orientation line 304 and a second orientation line 306 that is perpendicular to the first orientation line 304. The first orientation line 304 and the second orientation line 306 may be lines of symmetry of the first layer 202. A line of symmetry may be, for example, an imaginary line across the first side 206 or the second side 208 of the first layer 202 defining a fold line such that if the first layer 202 is folded on the line of symmetry, the through-holes 210 and the walls 212 on each side would be coincidentally aligned. Although the first layer 202 is shown as having a generally ovoid shape including longitudinal edges 308 and circular edges 310, the first layer 202 may have other shapes. For example, the first layer 202 may have a rectangular, diamond, square, circular, triangular, or amorphous shape. In some embodiments, the shape of the first layer 202 may be selected to accommodate the type of tissue site being treated. For example, the first layer 202 may have a circular shape to accommodate a circular tissue site. The first layer 202 may also be sizeable. For example, the first layer 202 may be cut, tom, or otherwise separated into portions to permit the first layer 202 to be diminished in size for smaller tissue sites. In some embodiments, the first orientation line 304 may be parallel to the longitudinal edges 308.

FIG. 4 is a detail view of a portion of the first layer 202 of FIG. 3, illustrating additional details that may be associated with some embodiments. The first layer 202 may include the plurality of through-holes 210 aligned in parallel rows to form an array. The array of through-holes 210 may include a first row 402 of the through-holes 210, a second row 404 of the through-holes 210, and a third row 406 of the through-holes 210. In some embodiments, a width 418 of the walls 212 between the perimeters 302 of adjacent through-holes 210 in a row, such as the first row 402, may be between about 2 and about 15 millimeters. In some embodiments, a width of about 5 millimeters may be preferred.

In some embodiments, a line connecting the centers 312 of adjacent rows may form a strut angle (SA) with the first orientation line 304. For example, a first through-hole 210A in the first row 402 may have a center 312A, and a second through-hole 210B in the second row 404 may have a center 312B. A strut line 412 may connect the center 312A with the center 312B. The strut line 412 may form an angle 414 with the first orientation line 304. The angle 414 may be the strut angle (SA) of the first layer 202. In some embodiments, the strut angle (SA) may be less than about 90°. In other embodiments, the strut angle (SA) may be between about 30° and about 70° relative to the first orientation line 304. In other embodiments, the strut angle (SA) may be about 66° from the first orientation line 304. Generally, as the strut angle (SA) decreases, a stiffness of the first layer 202 in a direction parallel to the first orientation line 304 may increase.

In some embodiments, the centers 312 of the through-holes 210 in alternating rows, for example, the center 312A of the first through-hole 210A in the first row 402 and a center 312C of a third through-hole 210C in the third row 406, may be spaced from each other parallel to the second orientation line 306 by a length 416. In some embodiments, the length 416 may be greater than an effective diameter of the through-hole 210. In some embodiments, the length 416 may be between about 2 mm and about 10 mm. If the centers 312 of through-holes 210 in alternating rows are separated by the length 416, an exterior surface of the walls 212 parallel to the first orientation line 304 may be considered continuous. Generally, the exterior surface of the walls 212 may be continuous if the exterior surface of the walls 212 do not have any discontinuities or breaks between through-holes 210.

In some embodiments, the through-holes 210 may be formed during molding of the first layer 202. In other embodiments, the through-holes 210 may be formed by cutting, melting, drilling, or vaporizing the first layer 202 after the first layer 202 is formed. For example, the through-holes 210 may be formed in the first layer 202 by laser cutting the compressed foam of the first layer 202.

In some embodiments, an effective diameter of the through-holes 210 may be selected to permit flow of particulates through the through-holes 210. In some embodiments, the through-holes 210 may have an average effective diameter between about 5 millimeters and about 20 millimeters. In some embodiments, a diameter of about 10 millimeters is preferred.

In some embodiments, the diameter of the through-holes 210 may be selected based on the size of the solubilized debris to be lifted from the tissue site. Larger through-holes 210 may allow larger debris to pass through the first layer 202, and smaller through-holes 210 may allow smaller debris to pass through the first layer 202 while blocking debris larger than the through-holes 210. In some embodiments, successive applications of the dressing 104 can use the first layers 202 having successively smaller diameters of the through-holes 210 as the size of the solubilized debris in the tissue site decreases. Sequentially decreasing diameters of the through-holes 210 may also aid in fine tuning a level of tissue disruption to the debris during the treatment of the tissue site. The diameter of the through-holes 210 can also influence fluid movement in the first layer 202 and the dressing 104. For example, the first layer 202 can channel fluid in the dressing 104 toward the through-holes 210 to aid in the disruption of the debris on the tissue site. Variation of the diameters of the through-holes 210 can vary how fluid is moved through the dressing 104 with respect to both the removal of fluid and the application of negative pressure.

In other embodiments, interior surfaces of the walls 212 of the plurality of through-holes 210 may be manufactured to prevent or reduce tissue granulation and tissue in-growth. For example, the plurality of through-holes 210 may be formed by thermoforming the first layer 202 causing the interior surfaces of the walls 212 to be smooth. As used herein, smoothness may refer to the formation of the through-holes 210 that causes the interior surfaces of the walls 212 that extend between the first side 206 and the second side 208. For example, laser-cutting the through-holes 210 into the first layer 202 may deform the material of the first layer 202, closing any pores on the interior surfaces of the walls 212 that extend between the first side 206 and the second side 208. In some embodiments, a smooth interior surface of the walls 212 may limit or inhibit ingrowth of tissue into the first layer 202 through the through-holes 210. In other embodiments, the smooth interior surfaces of the walls 212 may be formed by a smooth material or a smooth coating applied to the interior surfaces of the walls 212. In such embodiments, the plurality of perforations 216 in the second layer 204 may have a diameter substantially equal to a diameter of the plurality of through-holes 210 to allow slough and other fluids to be removed from the tissue site through the perforations 216 and the through-holes 210.

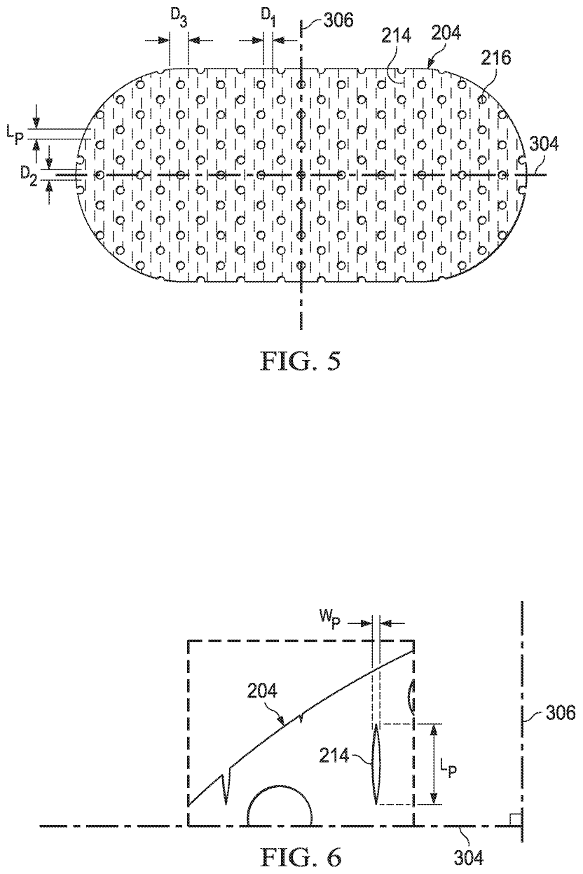

FIG. 5 is a plan view of the second layer 204 of FIG. 2, illustrating additional details that may be associated with some embodiments. The plurality of fluid restrictions 214 may be formed as slits or slots in the second layer 204. In some embodiments, the plurality of fluid restrictions 214 may be uniformly distributed in the second layer 204. For example, the fluid restrictions 214 may be substantially coextensive with the second layer 204 and be distributed across the second layer 204 in a grid of parallel rows and columns, in which the slots are also mutually parallel to each other. Adjacent rows of the fluid restriction 214 may be spaced a distance D₁, and the fluid restrictions 214 within each of the rows may be spaced a distance D₂. For example, a distance D₁ of about 3 millimeters on center and a distance D₂ of about 3 millimeters on center may be suitable for some embodiments. The fluid restriction 214 in adjacent rows may be aligned or offset. For example, adjacent rows of the fluid restrictions 214 may be offset, as illustrated in FIG. 5, so that the fluid restrictions 214 are aligned in alternating rows separated by a distance D₃. A distance D₃ of about 6 millimeters may be suitable for some examples. The spacing of the fluid restrictions 214 may vary in some embodiments to increase the density of fluid restrictions 214 according to therapeutic requirements.

Each of the plurality of perforations 216 disposed in the second layer 204 may have uniform or similar geometric properties. For example, in some embodiments, each of the plurality of perforations 216 may be circular and have substantially the same diameter. In some embodiments, each of the plurality of perforations 216 may have an effective diameter that is less than the effective diameter of each of the plurality of through-holes 210.

Although the second layer 204 is shown as having a generally ovoid shape, the second layer 204 may have other shapes. For example, the second layer 204 may have a rectangular, diamond, square, circular, triangular, or amorphous shape. In some embodiments, the shape of the second layer 204 may be selected to accommodate the type of tissue site being treated. For example, the second layer 204 may have a circular shape to accommodate a circular tissue site. The second layer 204 may be sizeable. For example, the second layer 204 may be cut, torn, or otherwise separated into portions to permit the second layer 204 to be diminished in size for smaller tissue sites. In some embodiments, the second layer 204 may be sized and shaped to substantially correspond to the size and shape of the first layer 202.

FIG. 6 is a detail view of a portion of the second layer 204 of FIG. 5, illustrating additional details that may be associated with some embodiments. The plurality of fluid restrictions 214 may be formed as slits or slots in the second layer 204. The plurality of fluid restrictions 214 may each consist essentially of one or more linear slots having a length L_{P} and a width W_{P}, wherein the length L_{P} extends parallel to the second orientation line 306 and the width W_{P} extends parallel to the first orientation line 304. In some embodiments, the plurality of fluid restrictions 214 may be linear slots having a length L_{P} less than 6 millimeters and a width W_{P} less than 2 millimeters. The length L_{P} may be at least 3 millimeters, and the width W_{P} may be at least 0.5 millimeters in some embodiments. A length L_{P} of about 3 millimeters and a width W_{P} of about 1 millimeter may be particularly suitable for many applications, and a tolerance of about 0.1 millimeter may be acceptable. The length L_{P} may be selected for particular applications, and a tolerance of about 0.1 millimeter may also be acceptable. Such dimensions and tolerances may be achieved with a laser cutter, for example. Slots of such configurations may function as imperfect elastomeric valves that can substantially reduce liquid flow in a normally closed or resting state. For example, such slots may form a flow restriction without being completely closed or sealed. The slots can expand or open wider in response to a pressure gradient and/or in response to the contraction of the first layer 202 to allow increased liquid flow.

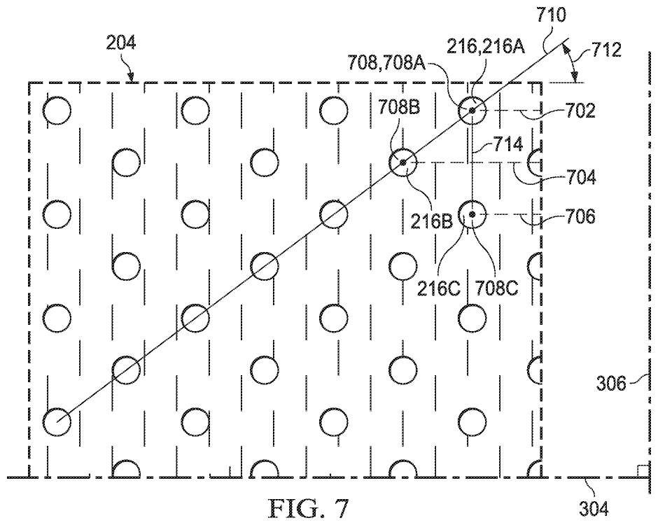

FIG. 7 is a detail view of a portion of the second layer 204 of FIG. 5, illustrating additional details that may be associated with some embodiments. The second layer 204 may include the plurality of perforations 216 aligned in parallel rows to form an array. The array of perforations 216 may include a first row 702 of the perforations 216, a second row 704 of the perforations 216, and a third row 706 of the perforations 216. Each of plurality of perforations 216 may have a center 708. The centers 708 of the perforations 216 in adjacent rows, for example, the first row 702 and the second row 704, may be characterized by being offset from the second orientation line 306 along the first orientation line 304. In some embodiments, the centers 708 of the perforations 216 in adjacent rows may be spaced between about 2 millimeters and about 6 millimeters. In some embodiments, the centers 708 of the perforations 216 in the second layer 204 may be configured to align with the centers 312 of the plurality of through-holes 210 in the first layer 202. For example, the centers 708 of the perforations 216 may be coincident with respective centers 312 of the plurality of through-holes 210.

In some embodiments, a line connecting the centers 708 of adjacent rows of the perforations 216 may form a strut angle (SA) with the first orientation line 304. For example, a first perforation 216A in the first row 702 may have a center 708A, and a second perforation 216B in the second row 704 may have a center 708B. A strut line 710 may connect the center 708A with the center 708B. The strut line 710 may form an angle 712 with the first orientation line 304. The angle 712 may be the strut angle (SA) of the second layer 204. In some embodiments, the strut angle (SA) may be less than about 90°. In other embodiments, the strut angle (SA) may be between about 30° and about 70° relative to the first orientation line 304. In other embodiments, the strut angle (SA) may be about 66° from the first orientation line 304.

In some embodiments, the centers 708 of the perforations 216 in alternating rows, for example, the center 708A of the first perforation 216A in the first row 702 and a center 708C of a third perforation 216C in the third row 706, may be spaced from each other parallel to the second orientation line 306 by a length 714. In some embodiments, the length 714 may be between about 4 mm and about 6 mm. In some embodiments, the length 714 may be greater than an effective diameter of the perforations 216.

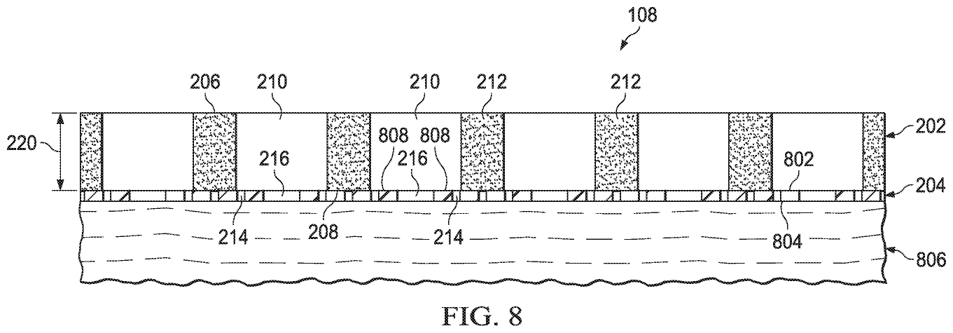

FIG. 8 is a cross-sectional view of the assembled tissue interface 108 of FIG. 2 taken along line 8-8, illustrating additional details that may be associated with some embodiments. The tissue interface 108 may include the first layer 202 and the second layer 204. In some embodiments, the second layer 204 may have a first side 802 configured to be coupled to the second side 208 of the first layer 202. For example, the first side 802 of the second layer 204 may be bonded, adhered, welded, or otherwise secured to the second side 208 of the first layer 202. In some embodiments, the second layer 204 may have a second side 804 opposite the first side 802 and configured to be positioned in direct contact with a tissue site 806.

The plurality of through-holes 210 may extend through the thickness 220 of the first layer 202 from the first side 206 to the second side 208. In some embodiments, the walls 212 may be parallel to the thickness 220 of the first layer 202. The plurality of perforations 216 may extend through the second layer 204 from the first side 802 to the second side 804 and have a periphery 808. In some embodiments, a center of each of the plurality of perforations 216 may be aligned with a center of each of the plurality of through-holes 210. Additionally, the diameter of each the plurality of perforations 216 may be less than the diameter of each of the through-holes 210. In some embodiments, the periphery 808 of each of the plurality of perforations 216 may be configured to extend into each of the through-holes 210 under an application of negative pressure and cover at least a portion of the walls 212, as discussed in more detail below.

FIG. 9 is a cross-sectional view of the assembled tissue interface 108 of FIG. 2 along line 8-8 during negative-pressure therapy, illustrating additional details that may be associated with some embodiments. The cover 110 may be positioned over the first layer 202 and the second layer 204 to create a sealed environment. FIG. 9 may illustrate a moment in time where a pressure in the sealed environment may be about −125 mm Hg of negative pressure. Negative pressure can generate a pressure gradient across the second layer 204 that concentrates stresses in the tissue site 806 adjacent the through-holes 210 in the first layer 202. The concentrated stresses can cause at least the periphery 808 of each of the plurality of perforations 216 of the second layer 204 to extend into each of the plurality of through-holes 210 of the first layer 202. In some embodiments, the periphery of each of the plurality of perforations 216 may be configured to completely cover the walls 212 to form a port. In other embodiments, the periphery of each of the plurality of perforations 216 may be configured to cover at least a portion of the walls 212. In some embodiments, the concentrated stresses can draw a portion of a subcutaneous tissue 902 at the tissue site 806 into the through-holes 210.

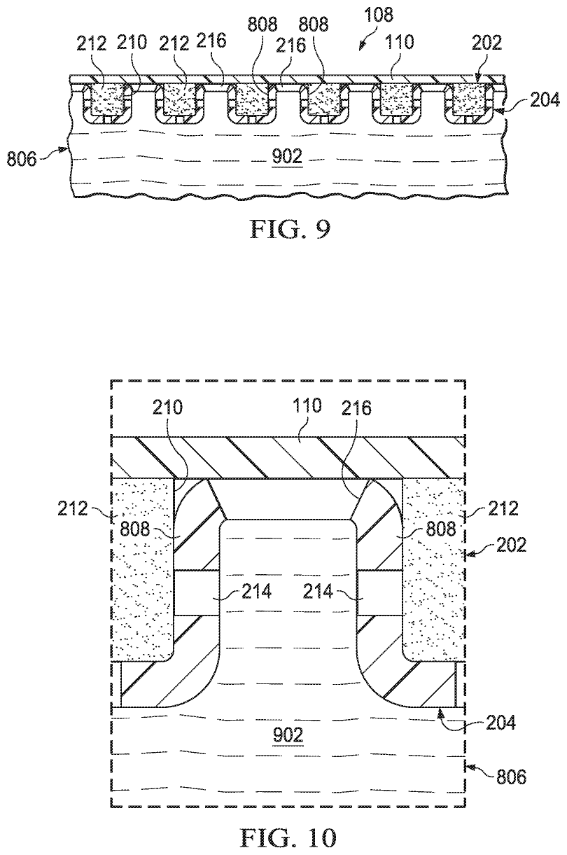

FIG. 10 is a detail view of a portion of the tissue interface 108 of FIG. 9, illustrating additional details of the tissue interface 108 during operation of negative-pressure therapy. Generally, the pressure in the sealed environment can exert a force that is proportional to the area over which the pressure is applied. In some embodiments, the force may be concentrated at the through-holes 210 of the first layer 202 and at the plurality of perforations 216 of the second layer 204 because there is less resistance to the application of negative pressure in the through-holes 210 and the plurality of perforations 216 than in the walls 212. In response to the force generated by the pressure at the through-holes 210 and the plurality of perforations 216, the periphery 808 of each of the plurality of perforations 216 may be drawn into and through each of the through-holes 210. The periphery 808 of each of the plurality of perforations 216 may cover at least a portion of an interior surface of the walls 212 of the through-holes 210 to create a port. In some embodiments, the periphery 808 of each of the plurality of perforations 216 may completely cover the interior surfaces of the walls 212 of the through-holes 210 to create the port. The port may allow thick slough and wound exudate to pass into and/or through the first layer 202. In some embodiments, at least a portion of the subcutaneous tissue 902 may be drawn into the through-holes 210. Additionally, the plurality of fluid restrictions 214 in the second layer 204 may allow fluids, such as wound exudate, to be removed from the tissue site 806 through the plurality of fluid restrictions 214 and into the first layer 202 under the application of negative pressure.

A height of the walls 212, the diameter of the through-holes 210, and the diameter of the perforations 216 may be selected to ensure that the periphery 808 of the plurality of perforations 216 in the second layer 204 may completely cover the walls 212 of the through-holes 210 to prevent tissue granulation and tissue in-growth. In some embodiments, the second layer 204 may be configured to stretch and the diameter of the plurality of perforations 216 may be selected to ensure the periphery 808 of each of the plurality of perforations 216 may stretch into the through-holes 210 and cover the walls 212.

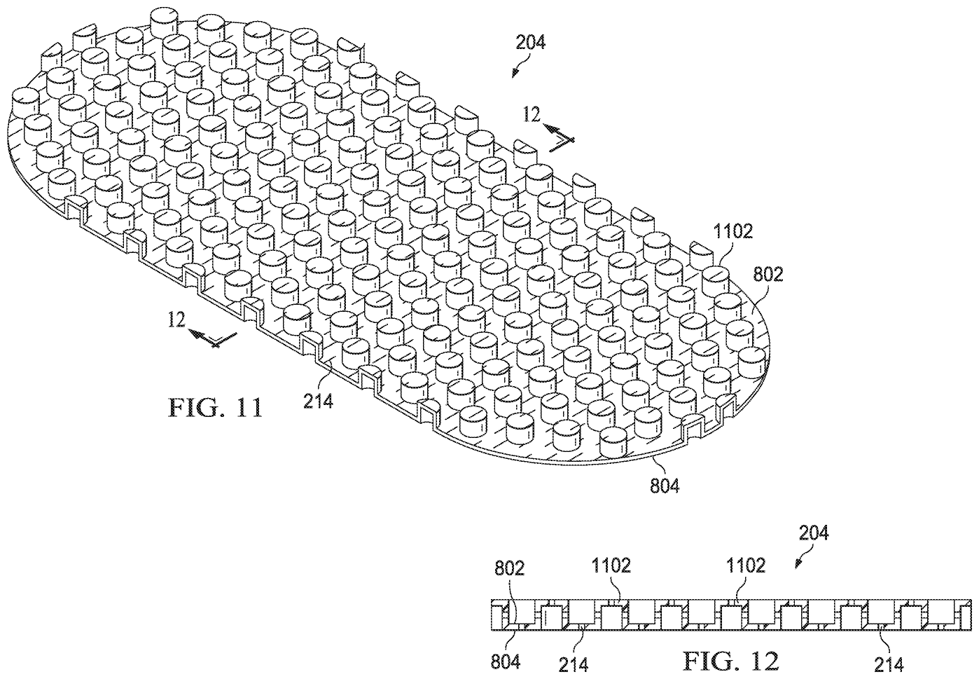

FIG. 11 is a perspective view of another example of the second layer 204 of FIG. 2, illustrating additional details that may be associated with some embodiments. In some embodiments, the second layer 204 may comprise the plurality of fluid restrictions 214 and a plurality of projections 1102. The plurality of projections 1102 may extend from the first side 802 of the second layer 205. In some embodiments, the plurality of projections 1102 may be thermoformed to create a cylindrical shape. In other embodiments, the plurality of projections 1102 may have other shapes and orientations, for example, elliptical, hexagonal, square, triangular, polygonal, or amorphous or irregular.

In some embodiments, each of the plurality of projections 1102 may correspond to one of the plurality of through-holes 210. For example, each of the plurality of projections 1102 of the second layer 204 may be configured to extend into at least a portion of one of the plurality of through holes 210 of the first layer 202. In other embodiments, there may be more through-holes 210 in the first layer 202 than there are projections 1102 in the second layer 204. For example, the plurality of projections 1102 may extend into a set of through-holes 210 and another set of through-holes 210 may not include any of the projections 1102.

In some embodiments, the plurality of projections 1102 may have a diameter substantially equal to the diameter of the through-holes 210. In other embodiments, the diameter of the plurality of projections 1102 may be smaller than the diameter of the through-holes 210.

In some embodiments, the plurality of projections 1102 in the second layer 204 may be configured to hold slough and other fluids removed from the tissue site within the projections 1102 while still preventing tissue granulation and tissue in-growth. Additionally, in some embodiments, the plurality of fluid restrictions 214 in the second layer 204 may be configured to release slough and other fluids into the first layer 202.

FIG. 12 is a cross-section view of the second layer 204 of FIG. 11 along line 12-12, illustrating additional details that may be associated with some embodiments. In some embodiments, the plurality of projections 1102 may have a substantially uniform height. In some embodiments, the plurality of projections 1102 may have a height equal to the thickness 220 of the first layer 202. In other embodiments, the height of the plurality of projections 1102 may be greater than the thickness 220 of the first layer 202. In still other embodiments, the height of the plurality of projections 1102 may be less than the thickness 220 of the first layer 202. For example, the plurality of projections 1102 may be configured to extend into a portion of the plurality of through-holes 210 of the first layer 202. Additionally or alternatively, the plurality of projections 1102 may have varying heights.

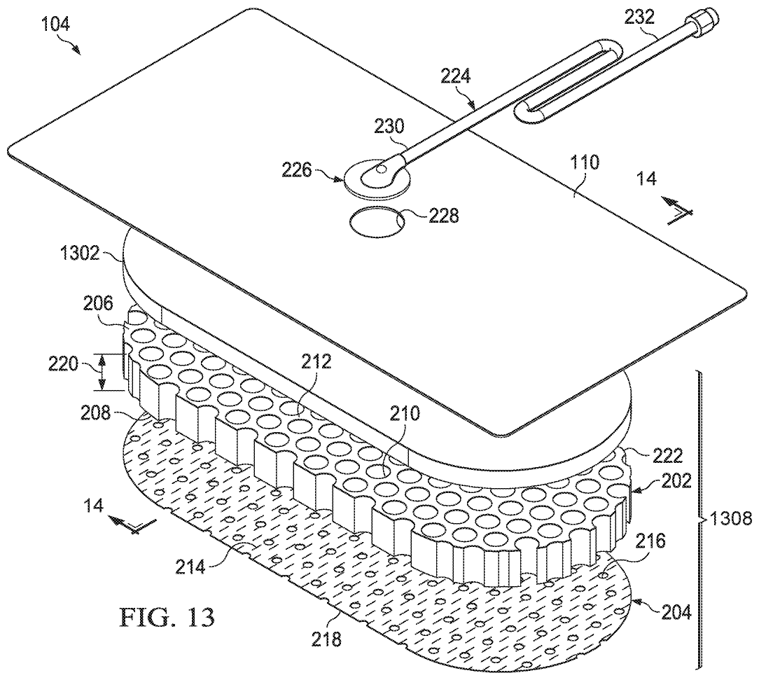

FIG. 13 is an assembly view of another example of the dressing 104 of FIG. 1, illustrating additional details that may be associated with some embodiments. The dressing 104 may include a tissue interface 1308 and the cover 104. The tissue interface 1308 may be similar to the tissue interface 108 and operate as described above with respect to FIGS. 2-8. For example, the tissue interface 1308 may include the first layer 202 and the second layer 204. In some embodiments, the tissue interface 1308 may also include a third layer 1302. The third layer 1302 may be configured to be coupled to the first side 206 of the first layer 202. The cover 110 may be configured to cover the third layer 1302. In some embodiments, the third layer 1302 may be a felted foam having a thickness less than or equal to the thickness 220 of the first layer 202.

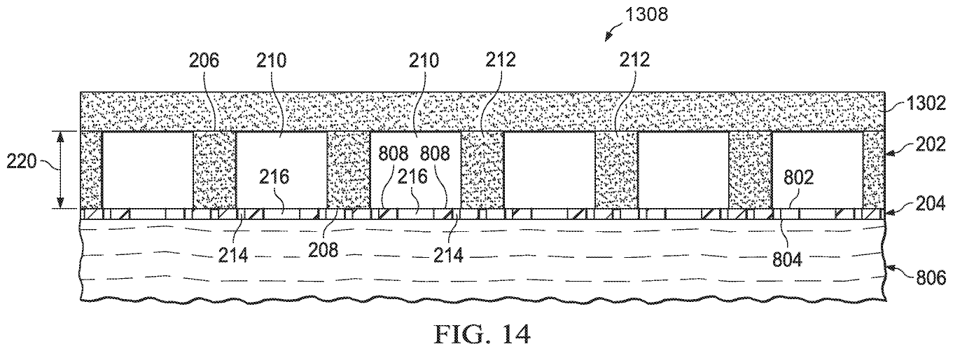

FIG. 14 is a cross-sectional view of the assembled tissue interface 1308 of FIG. 13 taken along line 14-14, illustrating additional details that may be associated with some embodiments. The tissue interface 108 may include the first layer 202, the second layer 204, and the third layer 1302. In some embodiments, the second layer 204 may have a first side 802 configured to be coupled to the second side 208 of the first layer 202. For example, the first side 802 of the second layer 204 may be bonded, adhered, welded, or otherwise secured to the second side 208 of the first layer 202. In some embodiments, the second layer 204 may have a second side 804 opposite the first side 802 and configured to be positioned in direct contact with a tissue site 806. In some embodiments, the third layer 1302 may be configured to be coupled to the first side 206 of the first layer 202.

In some embodiments, the plurality of through-holes 210 may extend through the thickness 220 of the first layer 202 from the first side 206 to the second side 208 and operate the same as discussed above with respect to FIGS. 2-10. In other embodiments, the first layer 202 and the third layer 1302 may be formed from a single layer of foam. In such embodiments, the through-holes 210 may comprise blind holes extending from the second side 208 of the second layer 202 toward the third layer 1302.

A method of manufacturing a dressing for a tissue site is also described herein. A first layer may be provided and a plurality of through-holes may be formed in the first layer. In some embodiments, the first layer may comprise a foam. In some embodiments, the method of forming a plurality of through-holes in the first layer may comprise die-cutting the foam to form a plurality of slugs and removing the plurality of slugs. In some embodiments, the method may further comprise felting the foam.

The method may further comprise providing a second layer. The second layer may be positioned adjacent to the first layer. In some embodiments, the second layer may be coupled to the first layer. For example, the second layer may be coupled to the first layer with an adhesive. A plurality of fluid restrictions and a plurality of perforations may be formed in the second layer. Forming the plurality of perforations in the second layer may comprise aligning the plurality of perforations with the plurality of through-holes in the first layer. Additionally or alternatively, forming the plurality of perforations in the second layer may comprise thermoforming a plurality of projections in the second layer configured to extend into the plurality of through-holes of the first layer. In some embodiments, the plurality of through-holes in the first layer and the plurality of projections in the second layer may comprise a shape that is circular, elliptical, polygonal, and/or amorphous.

Alternatively, other example embodiments may describe a system for treating a tissue site. The system may comprise a dressing, a sealing member configured to be positioned over the dressing and sealed to tissue surrounding the tissue site, and a reduced-pressure source configured to be fluidly coupled to the dressing through the sealing member. The dressing may comprise a debridement tool having a first side and a second side. In some embodiments, the debridement tool may have a plurality of openings extending through the debridement tool from the first side to the second side. In some embodiments, the debridement tool may comprise a foam. The dressing may also comprise a contact layer configured to be positioned adjacent the second side of the debridement tool. In some embodiments, the contact layer may have a plurality of fenestrations and a plurality of apertures disposed in the contact layer. The plurality of apertures may be configured to be aligned with the plurality of openings in the debridement tool. In some embodiments, the plurality of apertures may comprise a plurality of cylindrical projections configured to extend into the plurality of openings of the debridement tool.

In some embodiments, the contact layer may be coupled to the first side of the debridement tool. The contact layer may be coupled to the first side of the debridement tool with an adhesive. The adhesive may comprise a hot-melt adhesive. In some embodiments, the contact layer may comprise a polymer film. In some embodiments, the polymeric film may be hydrophilic. In some embodiments, the polymeric film may be hydrophobic.

A method of treating a tissue site is also described herein. A dressing can be positioned at the tissue site. The dressing can include a first layer having a first side and a second side and a second layer configured to be positioned adjacent the second side of the first layer. The first layer can include a plurality of through-holes extending through the first layer from the first side to the second side. The second layer can include a plurality of fluid restrictions and a plurality of perforations. The plurality of perforations can be configured to be aligned with the plurality of through-holes in the first layer. The method may further comprise fluidly coupling a negative-pressure source to the dressing; applying negative pressure from the negative-pressure source to the dressing; stretching a periphery of each of the plurality of perforations into the plurality of through-holes, creating a port in the first layer; and drawing fluid through the port. In some embodiments, drawing fluid through the port may comprise drawing fluid through the second layer into the first layer.

In some embodiments, the first layer may have a thickness extending from the first side to the second side. Each of the plurality of through-holes in the first layer may have side walls parallel to the thickness. Stretching the periphery of each of the plurality of perforations into the plurality of through-holes may comprise completely covering the side walls with the periphery of each of the plurality of perforations.

In some embodiments, the plurality of perforations in the second layer may comprise a plurality of cylindrical projections. In such embodiments, stretching the periphery of each of the plurality of perforations into the plurality of through-holes may comprise positioning each of the plurality of cylindrical projections in the plurality of through-holes. Additionally or alternatively, drawing fluid thorough the port may comprise collecting fluid in the plurality of cylindrical projections.

The systems, apparatuses, and methods described herein may provide significant advantages. For example, the second layer 204 may allow the dressing 104 to debride the tissue site while also reducing or preventing exposure of the tissue site 806 to the first layer 202, which can inhibit growth of tissue into the first layer 202. The periphery of each of the plurality of perforations 216 in the second layer 204 may stretch into the through-holes 210 and completely cover the walls 212 of the first layer 202 to create a port through each of the through-holes 210. Thick slough and wound exudate may be removed from the tissue site through each port while also preventing growth of tissue into the first layer 202 so that the dressing 104 can be worn for an extended period of time.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 104, the container 106, or both may be eliminated or separated from other components for manufacture or sale. In other example configurations, the controller 112 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A dressing for treating a tissue site with negative pressure, the dressing comprising:
a first layer comprising a first side and a second side;
a plurality of through-holes extending through the first layer from the first side to the second side;
a second layer configured to be positioned adjacent the second side of the first layer;
a plurality of fluid restrictions disposed in the second layer, and
a plurality of perforations disposed in the second layer and configured to be aligned with the plurality of through-holes.

2. The dressing of claim 1, wherein at least a periphery of each of the plurality of perforations is configured to extend into the plurality of through-holes in response to a pressure gradient across the second layer.

3. The dressing of claim 2, wherein the first layer has a thickness extending from the first side to the second side and each of the plurality of through-holes form side walls parallel to the thickness, and wherein the periphery of each of the plurality of perforations covers at least a portion of the side walls.

4. The dressing of claim 3, wherein the periphery of each of the plurality of perforations completely covers the side walls.

5. The dressing of claim 1, wherein each of the plurality of through-holes has an effective diameter between about 5 millimeters and about 20 millimeters.

6. The dressing of claim 1, wherein the plurality of through-holes comprise a first row of through-holes and a second row of through-holes along a longitudinal length of the first layer.

7. The dressing of claim 6, wherein one or more of the through-holes in the first row of through-holes are offset from the second row of through-holes.

8. The dressing of claim 1, wherein the plurality of through-holes are spaced between about 2 millimeters and about 10 millimeters on center.

9. The dressing of claim 1, wherein a portion of the plurality of fluid restrictions are registered with a portion of the plurality of through-holes.

10. The dressing of claim 1, wherein each of the plurality of perforations are smaller than each of the plurality of through-holes.

11. The dressing of claim 1, wherein each of the plurality of perforations are the same size as each of the plurality of through-holes.

12. The dressing of claim 1, wherein each of the plurality of perforations has an effective diameter less than the effective diameter of each of the plurality of through-holes.

13. The dressing of claim 1, wherein the plurality of perforations comprise a plurality of projections configured to extend into the plurality of through-holes.

14.-25. (canceled)

26. The dressing of claim 1, wherein the plurality of through-holes are circular, elliptical, polygonal, or amorphous.

27.-34. (canceled)

35. The dressing of claim 1, wherein the plurality of fluid restrictions comprise a plurality of slots, each of the slots having a length less than about 6 millimeters.

36. The dressing of claim 1, wherein the plurality of fluid restrictions comprise a plurality of slots, each of the slots having a width less than about 2 millimeters.

37. The dressing of claim 1, wherein the plurality of fluid restrictions comprise a plurality of fenestrations or a plurality of slits in the second layer.

38. (canceled)

39. The dressing of claim 1, wherein the second layer is configured to be positioned in direct contact with the tissue site.

40. The dressing of claim 1, wherein the second layer is coupled to the second side of the first layer.

41. The dressing of claim 1, further comprising a cover configured to create a sealed space containing the first layer and the second layer at the tissue site.

42.-68. (canceled)
